(19) United States
(12) Patent Application Publication
(10) Pub. No.: US 2019/0216661 A1
(43) Pub. Date: Jul. 18, 2019

(54) WHEELCHAIR USER SUPPORT MAPPING SYSTEM

(71) Applicant: HONDA MOTOR CO., LTD., Tokyo (JP)

(72) Inventors: Masayo ARAI, Wako-shi (JP); Takamasa KOSHIZEN, Wako-shi (JP)

(21) Appl. No.: 16/248,183

(22) Filed: Jan. 15, 2019

(30) Foreign Application Priority Data
Jan. 17, 2018 (JP) 2018-005488

(51) Int. Cl. A61G 5/10; G06N 20/00; G01C 21/20

(52) U.S. Cl. CPC A61G 5/10; G06N 20/00; A61G 5/06; A61G 2203/70; G01C 21/20

(57) ABSTRACT

A wheelchair user support mapping system includes: an association unit (barrier information database) configured to store actual image data of a location corresponding to a predetermined position on a map in such a way as to be capable of outputting the image data while associating the image data with the predetermined position on the map; an action history storage unit (individual barrier condition database) configured to extract and store a barrier condition, which constitutes a criterion for passability and impassability, based on an action history of a wheelchair user; and a movement plan creation unit (recommended route computation unit) configured to create a movement plan for the wheelchair user based on the barrier condition acquired with reference to the action history storage unit.

### CROSS-REFERENCE TO RELATED APPLICATION

This application is based upon and claims priority from the Japanese Patent Application No. 2018-005488, filed on Jan. 17, 2018, the entire contents of which are incorporated herein by reference.

### BACKGROUND OF THE INVENTION

### 1. Field of the Invention

The present invention relates to a wheelchair user support mapping system.

### 2. Description of the Related Art

A mapping system has been known which is configured to display a route having a high passage frequency as a recommended route while overwriting such a route travelled by wheelchair users on a map in a database (see Japanese Patent Application Publication No. 2003-240592 (Patent Document 1), for example).

This mapping system allows another wheelchair user to presume that the recommended route displayed on the system is the route having the high passage frequently and is therefore probably barrier-free. In other words, the wheelchair user would naturally presume that he or she can pass through the displayed recommended route smoothly on a wheelchair.

In fact, however, it is not possible to determine barrier conditions of routes of passage, that is, degrees of barriers (such as level differences and slopes) constituting criteria for passability and impassability to be universally applicable to all wheelchair users. In this context, a recommended route according to the conventional mapping system (see Patent Document 1, for example) may be a passable route for a certain wheelchair user but maybe an impassable route for another wheelchair user. On the other hand, depending on the degrees of the barriers, there may be a case where a wheelchair user escorted by a helper is able to pass through a route having a low passage frequency (a non-recommended route) according to the mapping system (see Patent Document 1, for example).

The present invention has therefore been made in view of the above problem, and an object of the invention is to provide a wheelchair user support mapping system capable of displaying an optimum passage route tailored to individual wheelchair users.

### SUMMARY OF THE INVENTION

In order to solve the above problem, according to an aspect of the present invention, a wheelchair user support mapping system reflecting one aspect of the present invention includes: an association unit configured to store actual image data of a location corresponding to a predetermined position on a map in such a way as to be capable of outputting the image data while associating the image data with the predetermined position on the map; an action history storage unit configured to extract and store a barrier condition, which constitutes a criterion for passability and impassability, based on an action history of a wheelchair user; and a movement plan creation unit configured to create a movement plan for the wheelchair user based on the barrier condition acquired with reference to the action history storage unit.

### BRIEF DESCRIPTION OF THE DRAWINGS

The features and advantages provided by one or more embodiments of the invention will become apparent from the detailed description given below and appended drawings which are given only by way of illustration, and thus are not intended as a definition of the limits of the present invention.

FIG. 1 is an explanatory configuration diagram of a wheelchair user support mapping system according to an embodiment of the present invention.

FIG. 2 is a block diagram of a cloud system constituting the wheelchair user support mapping system of FIG. 1.

FIG. 3 is an operation flowchart for outputting a recommended route by the wheelchair user support mapping system of FIG. 1.

FIG. 4 is a flowchart of a recommended route computation step to be executed by a movement plan creation unit constituting the cloud system of FIG. 2.

FIG. 5 is a diagram of an image of barrier information associated with a map and stored in an association unit.

FIG. 6 is a map containing a recommended route displayed on a display unit constituting the wheelchair user support mapping system of FIG. 1.

FIG. 7 is a map obtained by combining display of a shot image of a barrier being a cause of impassability with the map of FIG. 6.

FIGS. 8A to 8C are diagrams of images on the display unit showing the progress from input of a point of departure, a point of destination, and a pass point to output of a recommended route.

FIG. 9 is a diagram of an image on the display unit showing an aspect in which the recommended route is output after the point of departure, the point of destination, a first pass point, and a second pass point are input.

FIG. 10 is an explanatory configuration diagram of the wheelchair user support mapping system, which feeds back evaluations of the recommended route by users as action histories of a wheelchair user.

FIG. 11 is a diagram showing an example of individual barrier conditions to be accumulated in an individual barrier condition database.

### DETAILED DESCRIPTION OF THE PREFERRED EMBODIMENTS

One or more embodiments of the present invention will be hereinafter described in detail with reference to the drawings as necessary.

A wheelchair user support mapping system of a mode to carry out (an embodiment of) the present invention will be described in detail.

The wheelchair user support mapping system of this embodiment is configured to support a wheelchair user by offering a route of passage (a recommended route) for bypassing barriers, which are obstacles to passage of the wheelchair user, in answer to input of a point of destination and a point of destination by the wheelchair user.

Specifically, the wheelchair user support mapping system outputs a recommended route based on barrier conditions applicable to an individual wheelchair user. In other words, this wheelchair user support mapping system is widely available to multiple wheelchair users and yet offers an optimum recommended route tailored to the individual wheelchair user who requests the recommended route.

In addition, the wheelchair user support mapping system is configured to display an image of a barrier (a barrier image) being a cause of exclusion of a route containing the barrier from route candidates for the recommended route, a text and/or an image constituting a reason or a basis of selection of the recommended route, and so forth. Note that the barrier conditions of this embodiment are degrees of barriers against the individual wheelchair user which constitute criteria for passability and impassability. The barrier conditions will be described in detail later.

<Configuration of Wheelchair User Support Mapping System>

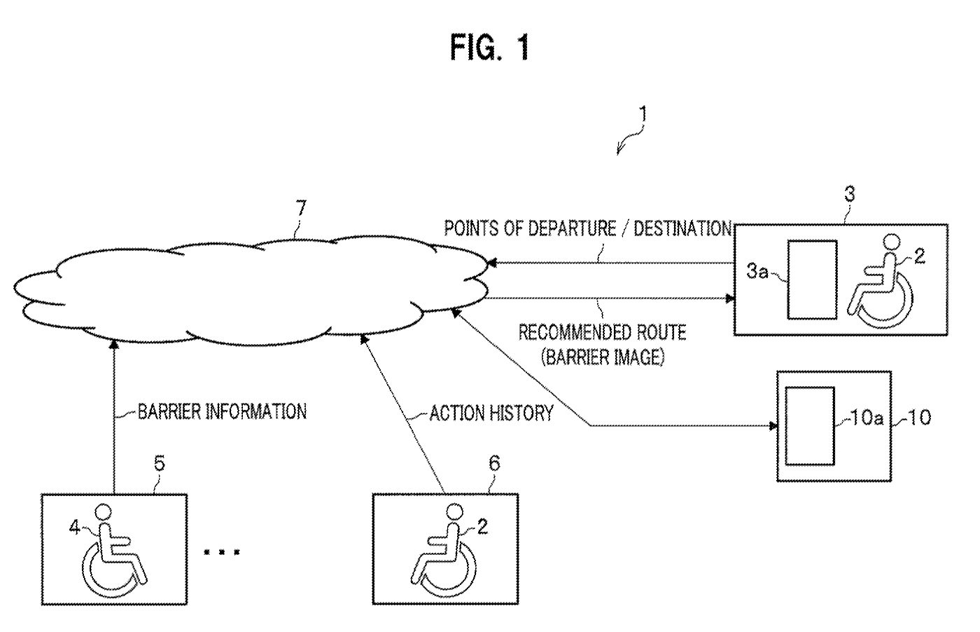

FIG. 1 is an explanatory configuration diagram of a wheelchair user support mapping system 1 of this embodiment.

As shown in FIG. 1, the wheelchair user support mapping system 1 includes: a first mobile terminal 3 that belongs to a wheelchair user 2 who requests an offer of a recommended route; multiple second mobile terminals 5 owned by multiple wheelchair users 4, respectively, and configured to transmit a variety of information on barriers which are general obstacles to passage of wheelchair users (hereinafter simply referred to as “barrier information”); a third mobile terminal 6 configured to transmit individual action histories (barrier conditions) of the wheelchair user 2 who requests the offer of the recommended route; and a cloud system 7 configured to compute and output the recommended route based on the barrier information and the barrier conditions described above in response to a request for the recommended route by the wheelchair user 2.

Moreover, the wheelchair user support mapping system 1 of this embodiment may also include a fixed terminal 10 configured to communicate with the cloud system 7 as described in detail later.

Here, the only difference between the first mobile terminal 3 and the third mobile terminal 6 of the wheelchair user 2 lies in that the first mobile terminal 3 is configured to receive the offer of the recommended route from the cloud system 7 whereas the third mobile terminal 6 is configured to output information (the action histories of the wheelchair user 2) used for the computation of the recommended route to the cloud system 7. In this context, the first mobile terminal 3 and the third mobile terminal 6 may be incorporated into a single mobile terminal owned by the wheelchair user 2 as long as the single mobile terminal has functions of the respective terminals to be described later.

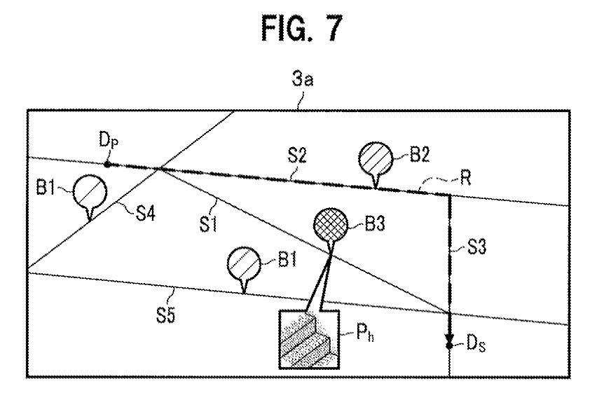

The configuration of the first mobile terminal 3 is not limited as long as the first mobile terminal 3 is capable of requesting the offer of the recommended route from the cloud system 7 and displaying the recommended route offered from the cloud system 7. Specifically, the first mobile terminal 3 is assumed to have a display unit 3a, which is capable of sending the cloud system 7 a point of departure and a point of destination, and is configured to display the recommended route and a barrier image Ph (see FIG. 7) to be described later, which are transmitted from the cloud system 7.

The display unit 3a corresponds to “a display unit configured to display the movement plan and the image data to the wheelchair user after the wheelchair user actually starts a movement” as defined in the appended claim.

Examples of the first mobile terminal 3 include a smartphone, a tablet, a laptop personal computer, and the like. Among them, the smartphone is particularly preferable because of its excellent portability.

Here, assuming that the first mobile terminal 3 is any of the smartphone, the tablet, and the laptop personal computer, for example, the input of the point of departure and the point of destination to the cloud system 7 can be easily achieved by utilizing an API (application programming interface) disclosed by an OS (operating system) for the first mobile terminal 3.

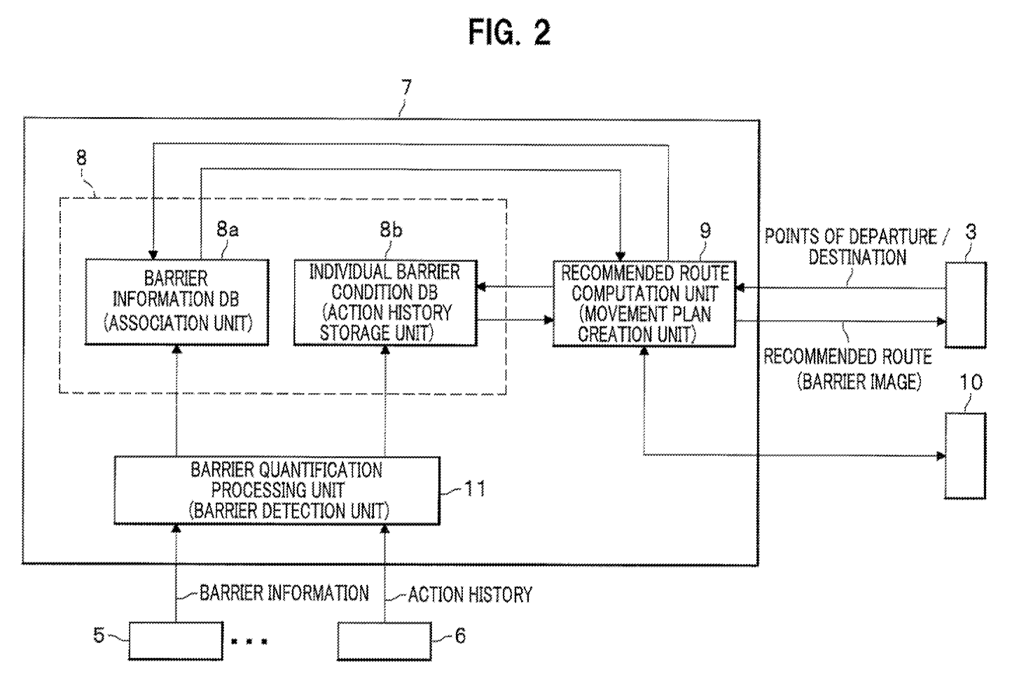

Each second mobile terminal 5 transmits the above-described barrier information to a barrier quantification processing unit 11 of the cloud system 7 to be described later (see FIG. 2).

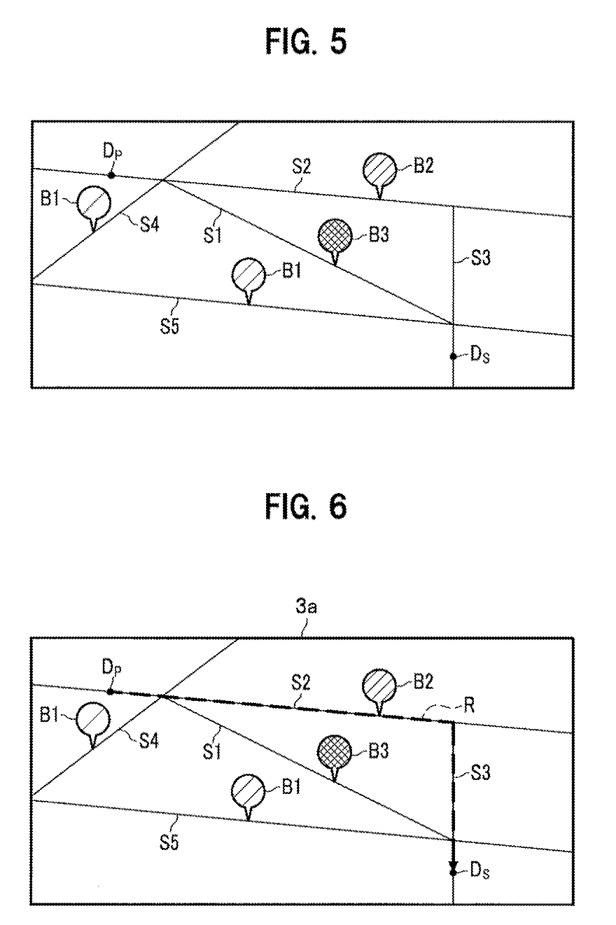

Note that the barrier information of this embodiment is assumed to be provided from the multiple wheelchair users 4 who have actually passed through a predetermined area (such as an area illustrated with a map of FIG. 5 to be described in detail later). Each piece of the barrier information is mainly formed from image data of a barrier shot by the each wheelchair user 4 and information (coordinate data) on a position where the barrier is present.

Incidentally, the map of FIG. 5 of this embodiment coincides with an area for which the wheelchair user 2 requests the recommended route. Nonetheless, the predetermined area on which the wheelchair user 2 is provided with the barrier information is not limited to the area of the map of FIG. 5 but is supposed to encompass the entire areas where the wheelchair user support mapping system 1 is deployed.

Each second mobile terminal 5 of this embodiment configured to output the above-described barrier information is equipped with a camera for shooting barrier images and a GPS (global positioning system) function. In this context, the second mobile terminal 5 may be any of a smartphone, a tablet, and a laptop personal computer as described above as long as the terminal is equipped with the image shooting camera and the GPS function.

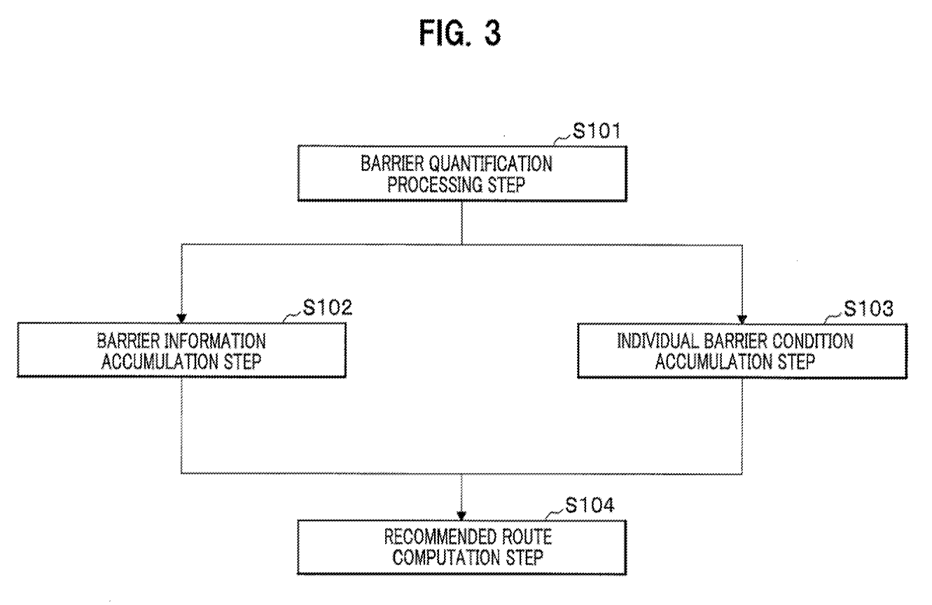

The third mobile terminal 6 transmits individual action histories of the wheelchair user 2 to the barrier quantification processing unit 11 (see FIG. 2) to be described later, of the cloud system 7. The action histories are used in a step of extracting barrier conditions applicable to the wheelchair user 2 (an individual barrier condition accumulation step S103 (see FIG. 3) to be described later).

The action history is mainly formed from shot image data of barriers shot by the wheelchair user 2 based on conditions representing passability and impassability of the predetermined area, and information (coordinate data) on a position where the barrier is present. Moreover, when such a barrier is a state of unevenness, a level difference, or the like of a road surface, the corresponding piece of data of the action history is obtained by adding undulation (acceleration) data, which is acquired at the time of passage on this road surface with a wheelchair, to the shot image data of the road surface.

Note that each action history of this embodiment is provided from the wheelchair user 2 who actually passes through the area where the wheelchair user support mapping system 1 is deployed.

The third mobile terminal 6 of this embodiment configured to output the above-described action history may be any of a smartphone, a tablet, and a laptop personal computer as long as the terminal is equipped with the shooting camera, the GPS function, a vibrometer (an accelerometer), and the like.

The fixed terminal 10 of this embodiment is assumed to be available not only for the wheelchair user 2 but also for a person other than the wheelchair user 2.

The fixed terminal 10 is assumed to be a fixed terminal located at the home or the like of the wheelchair user 2 for private use of the wheelchair user 2, or a terminal located in a public space for free use by many and unspecified persons, for example.

The fixed terminal 10 is not limited to a particular configuration as long as the terminal is capable of requesting the cloud system 7 to offer the recommended route and displaying the recommended route offered from the cloud system 7. A typical example of the fixed terminal 10 is a desktop personal computer, which is capable of transmitting the point of departure and the point of destination to the cloud system 7, and is provided with a display unit 10a configured to display the recommended route and the barrier image Ph (see FIG. 7) to be described later, which are transmitted from the cloud system 7.

Note that the display unit 10a corresponds to a “display unit configured to display the movement plan and the image data in advance before the wheelchair user starts a movement” as defined in the appended claim.

Next, the cloud system 7 will be described.

FIG. 2 is a block diagram of the cloud system 7 of this embodiment.

As shown in FIG. 2, the cloud system 7 includes a barrier information DB (database) 8a serving as an association unit and an individual barrier condition DB (database) 8b serving as an action history storage unit, which collectively constitute a DB (database) 8, and a recommended route computation unit 9 serving as a movement plan creation unit that computes the recommended route based on the barrier information and the barrier conditions stored in the DB 8. In addition, the cloud system 7 further includes a barrier quantification processing unit 11 serving as a barrier detection unit. Note that reference sign 10 in FIG. 2 denotes the above-described fixed terminal.

Description will be first given of the barrier quantification processing unit 11 (the barrier detection unit).

The barrier quantification processing unit 11 is configured to subject the barrier information (the image data shot with the cameras) transmitted from the second mobile terminals 5 to classification processing by means of image determination to be described later.

Moreover, the barrier quantification processing unit 11 is configured to subject the action histories (the image data shot with the camera) transmitted from the third mobile terminal 6 to the classification processing by means of the image determination to be described later. The various barrier conditions to be described later, applicable to the wheelchair user 2, are set in this way.

The barrier information DB 8a (the association unit) is configured to accumulate pieces of the barrier information classified by the barrier quantification processing unit 11 (the barrier detection unit) while associating each piece of the information with position information (coordinate data) on the corresponding barrier. Moreover, the barrier information DB 8a (the association unit) is also configured to accumulate the images (the barrier images) shot with the second mobile terminal 5 and subjected to the image classification while associating each shot image with the position information (the coordinate data).

The individual barrier condition DB 8b (the action history storage unit) is configured to accumulate the action histories (the barrier conditions) classified by the barrier quantification processing unit 11 (the barrier detection unit) together with distinctions between passability and impassability.

The recommended route computation unit 9 (the movement plan creation unit) is configured to compute and output the recommended route as described later by referring to the barrier information accumulated in the barrier information DB 8a (the association unit) and the action histories (the barrier conditions) of the wheelchair user 2 accumulated in the individual barrier condition DB 8b (the action history storage unit).

<Operation Procedures of Wheelchair User Support Mapping System>

FIG. 3 is an operation flowchart for outputting the recommended route by the wheelchair user support mapping system 1 of FIG. 1.

Before explaining a recommended route computation step S104 (see FIG. 3) to be executed by the recommended route computation unit 9 (see FIG. 2) serving as the movement plan creation unit, a description will be given below of a barrier quantification processing step S101 (see FIG. 3), a barrier information accumulation step S102 (see FIG. 3), and an individual barrier condition accumulation step S103 (see FIG. 3).

The barrier quantification processing step S101 shown in FIG. 3 is executed by the barrier quantification processing unit 11 (see FIG. 2) serving as the barrier detection unit.

In the barrier quantification processing step S101, the classification processing by means of the image determination is performed on the barrier information from the second mobile terminals 5 (see FIG. 2) and on the action histories from the third mobile terminal 6 (see FIG. 2) as described above.

The image data as the barrier information from the second mobile terminals 5 are subjected to classification depending on the attributes to be described later, such as road widths of pathways during passage through the predetermined area by the wheelchair users 4 (see FIG. 1) together with degrees (intensities) of the attributes by means of the image determination. The above-described image determination is executed by machine learning that uses an image determination unit having a publicly known structure.

While the image determination by the machine learning can be implemented by using a publicly known algorithm, the image determination of this embodiment is assumed to use deep learning in light of classification accuracy. Specifically, this embodiment assumes the image determination unit which uses a convolutional neutral network (CNN).

Nonetheless, this embodiment is not limited to the above-described image determination. For example, it is also possible to adopt a method of defining a shape constituting a base to be included in an image and conducting classification depending on whether or not there is the base shape in a determination target image. Moreover, in this embodiment, it is also possible to adopt a method of calculating feature vectors that contain gradient moments as elements, which are products of pixel value gradients and coordinate values of an image, and conducting classification based on similarity to a result obtained by the machine learning while using at least one of a known image and a newly acquired image. In other words, the image determination of this embodiment is based on a concept of using a model which is present from the beginning and on a concept of constructing a model from scratch.

The image data as the action histories from the third mobile terminal 6 are subjected to classification depending on the attributes to be described later, such as road widths of pathways during passage through the predetermined area by the wheelchair user 2 together with the degrees (the intensities) of the attributes by means of the image determination. Each classified attribute is provided with a distinction as to whether the attribute renders the wheelchair user 2 passable or impassable. Moreover, if an attribute concerns a level difference or a road surface condition (the degree of unevenness) as described later, the attribute is provided with the undulation (acceleration) data during the passage of the road surface.

The barrier information accumulation step S102 is executed by the barrier information DB 8a (see FIG. 2) serving as the association unit.

In the barrier information accumulation step S102, the classified pieces of the barrier information are accumulated in the barrier information DB 8a together with the degrees (the intensities) of the respective attributes thereof in such a way as to be associated with a map of the predetermined area traveled by the wheelchair users 4 (see FIG. 1) based on the position information (the coordinate data) constituting the pieces of the barrier information.

An open API service using the Web GIS (geographic information system) (such as the Ajax of the Google Map (registered trademark) API) can be used as the map of the predetermined area.

Incidentally, a range of the predetermined area is preferably expanded not only to domestic areas but also to foreign areas.

The individual barrier condition accumulation step S103 is executed by the individual barrier condition DB 8b (see FIG. 2) serving as the action history storage unit.

In the individual barrier condition accumulation step S103, individual barrier conditions being applicable to the wheelchair user 2 and constituting criteria for determining whether given barriers in the predetermined area, for which the wheelchair user 2 requests the recommended route, render the wheelchair user 2 passable or impassable are accumulated in the individual barrier condition DB 8b together with the degrees (the intensities) of the respective attributes thereof.

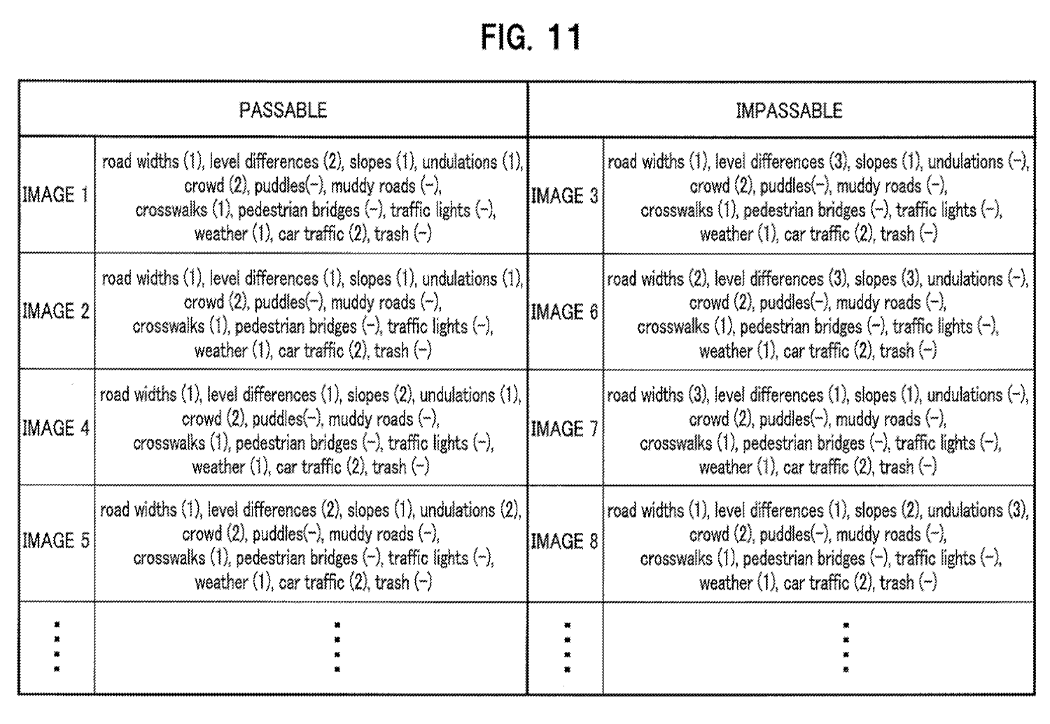

FIG. 11 shows an example of the individual barrier conditions to be accumulated in the individual barrier condition DB 8b.

The individual barrier conditions shown in FIG. 11 include thirteen attributes to be classified in the barrier quantification processing step S101 (see FIG. 3), namely, whether a road width of a pathway is wide or narrow, whether a level difference thereon is large or small, whether a slope thereof is large or small, where undulations thereon are large or small (the degree of unevenness on the road surface), whether crowd thereon is large or small, presence or absence of puddles, presence or absence of muddy road parts, presence or absence of crosswalks, presence or absence of pedestrian bridges, presence or absence of traffic lights, a weather condition (good weather or bad weather), whether car traffic thereon is busy or not, and whether or not there are many trash collection sites thereon. Needless to say, the types and the number of the attributes are not limited to the foregoing.

Moreover, images 1 to 8 in FIG. 11 correspond to respective pieces of the image data representing the barrier information from the third mobile terminal 6 (see FIG. 2). Moreover, a parenthesized number suffixed to each attribute indicates the degree (the intensity) of the attribute. To be more precise, the degree (the intensity) of the attribute may be defined like (1) as being very easily passable, (2) as being fairly passable, and (3) as being impassable, for example. Moreover, a suffix (−) attached to any of the attributes indicates that the relevant attribute is not present.

While the numerical value indicating the degree (the intensity) of each attribute is subjectively determined by the wheelchair user 2, this numerical value is associated with the degree (the intensity) of the corresponding attribute determined at the time of the image determination by the machine learning in the above-described barrier quantification processing step S101. Accordingly, the numerical value indicating the degree (the intensity) of the attribute, which is subjectively determined by the wheelchair user 2, is also associated with the degree (the intensity) of the corresponding attribute of the barrier information accumulated in the barrier information DB 8a in the barrier information accumulation step S102. In this instance, each barrier which is included in every piece of the image data representing the action history and formed into the data based the degree of the barrier, is defined as training data.

The recommended route computation step S104 is executed by the recommended route computation unit 9 (see FIG. 2) serving as the movement plan creation unit.

As shown in FIG. 1, the recommended route computation step S104 is executed by causing the wheelchair user 2 to transmit the point of departure and the point of destination to the cloud system 7 through the first mobile terminal 3.

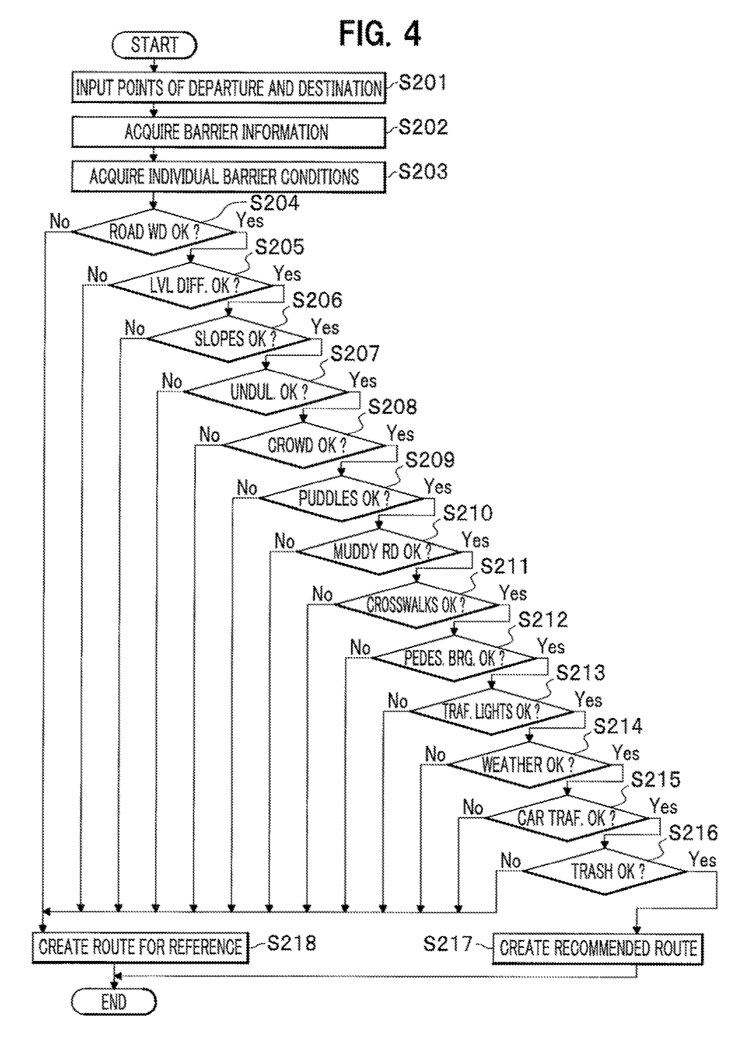

FIG. 4 is a flowchart of the recommended route computation step S104 (see FIG. 3) to be executed by the cloud system 7.

As shown in FIG. 4, in response to the input of the point of departure and the point of destination by the wheelchair user 2 (see FIG. 1) (step S201), the recommended route computation unit 9 (see FIG. 2) refers to the barrier information DB 8a (the association unit). In this way, the recommended route computation unit 9 acquires the barrier information including the point of departure and the point of destination in the form of the coordinate data (step S202). As described above, the barrier information is accumulated in the barrier information DB 8a while being associated with the map of the predetermined area.

FIG. 5 is a diagram of an image of the barrier information stored in the barrier information DB 8a while being associated with the map.

In FIG. 5, reference signs S1 to S5 denote pathways in the area indicated with the map. Reference sign D_{P} denotes the point of departure input by the wheelchair user 2, and reference sign D_{S} denotes the point of destination input by the wheelchair user 2.

The recommended route computation unit 9 computes the following route candidates from the point of departure D_{P} to the point of destination D_{S} based on the barrier information shown in FIG. 5 that is acquired from the barrier information DB 8a, namely, a route that passes through the pathway S1, a route that passes through the pathways S2 and S3, and a route that passes through the pathways S4 and S5.

Moreover, the recommended route computation unit 9 identifies four barriers B1, B1, B2, and B3 present on the route candidates based on the acquired barrier information shown in FIG. 5.

Incidentally, the barrier B1 represents the one in which all the attributes have the degrees (the intensities) equivalent to (1). Moreover, the barrier B2 represents the one in which at least one of the attributes has the degree (the intensity) equivalent to (2) while none of the attributes has the degree (the intensity) equivalent to (3). The barrier B3 represents the one in which at least one of the attributes has the degree (the intensity) equivalent to (3).

Referring back to FIG. 4, the recommended route computation unit 9 refers to the individual barrier condition DB 8b (see FIG. 2) and acquires the individual barrier conditions (step S203).

More specifically, the recommended route computation unit 9 refers to the individual barrier conditions shown in FIG. 11, for example, and estimates that the route candidate is impassable if at least one of the attributes has the degree (the intensity) equivalent to (3), and estimates that the route candidate is passable in any other case.

In other words, when all of the above-described attributes have the degrees (the intensities) below the predetermined values (when all outcomes of steps S204 to S216 are yes), the recommended route computation unit 9 creates the recommended route by selecting the route candidate that satisfies the above-described conditions out of all of the route candidates (step S217).

On the other hand, if these conditions are not satisfied (when all the outcomes of steps S204 to S216 are no), the recommended route computation unit 9 outputs a predetermined number of routes in step S218 as routes for reference in ascending order of the degrees (the intensities) of the attributes therein. More specifically, the recommended route computation unit 9 outputs the routes for reference having fewer barriers B3.

Here, if there are two or more route candidates, then it is possible to select the route with the shortest distance or to select the route in which the attributes constituting the route have the degrees (the intensities) that are relatively low. Moreover, it is possible to set only one recommended route or to set two or more recommended routes.

Then, as a consequence of the output of the recommended route (or the routes for reference) from the recommended route computation unit 9, the map indicating the recommended route (or the routes for reference) is displayed on the display unit 3a (see FIG. 1) of the first mobile terminal 3 (see FIG. 1) and on the display unit 10a (see FIG. 1) of the fixed terminal 10 (see FIG. 1).

FIGS. 6 and 7 show a map containing a recommended route R and being displayed on the display units 3a and 10a.

As shown in FIG. 6, the display units 3a and 10a display the map indicating the recommended route R, which connects the point of departure DP and the point of destination DS at the shortest distance while bypassing the impassable barrier B3.

Moreover, by touching an icon (or pointing the icon with a cursor and clicking the icon) indicating the barrier B3 on the display units 3a and 10a, the barrier image Ph being the cause of impassability is displayed together as shown in FIG. 7.

In FIGS. 6 and 7, reference signs S1 to S5 denote the pathways while reference signs B1 and B2 denote the barriers which the wheelchair user 2 can pass through.

<Operation and Effects>

Next, description will be given of operation and effects to be obtained from the wheelchair user support mapping system according to the present embodiment.

As described above, a recommended route according to the conventional mapping system (see Patent Document 1, for example) may be a passable route for a certain wheelchair user but may be an impassable route for another wheelchair user.

In contrast, the wheelchair user support mapping system 1 (see FIG. 1) of this embodiment outputs the recommended route R (see FIG. 6) based on the barrier conditions applicable to the individual wheelchair user (which are the barrier conditions applicable to the wheelchair user 2 (see FIG. 1) in this embodiment).

According to the above-described wheelchair user support mapping system 1, it is possible to output the optimum recommended route R which is tailored solely to the wheelchair user 2 who requests the recommended route R.

As shown in FIG. 1, the wheelchair user support mapping system 1 of this embodiment includes the barrier information DB 8a (the association unit) configured to store the actual image data (the image shot with the second mobile terminal 5) of the location corresponding to the predetermined position on the map in such a way as to be capable of outputting the image data while associating the image data with the predetermined position on the map. Moreover, the wheelchair user support mapping system 1 includes the individual barrier condition DB (the action history storage unit) configured to extract and store the predetermined barrier conditions, which are applicable to the individual wheelchair user 2 and constitute the criteria for passability and impassability, based on the action histories of the wheelchair user 2. Furthermore, the wheelchair user support mapping system 1 includes the recommended route computation unit 9 (the movement plan creation unit) configured to create the movement plan for the wheelchair user 2 based on the barrier conditions acquired with reference to the individual barrier condition DB.

The wheelchair user support mapping system 1 creates the map associated with the actual shot image and creates the recommended route (the movement plan) based on the predetermined barrier conditions.

The above-described wheelchair user support mapping system 1 is capable of allowing the wheelchair user 2 to confirm the types of the barriers by oneself, and creating the recommended route (the movement plan) that matches environments involving the wheelchair user 2 (the physical strength and condition of the wheelchair user, mechanical conditions of the electric or non-electric wheelchair, and so forth).

Moreover, the above-described wheelchair user support mapping system 1 can develop the recommended route (the movement plane) that matches the above-described environments involving the wheelchair user 2 more precisely.

Moreover, the above-described wheelchair user support mapping system 1 includes the barrier quantification processing unit 11 (the barrier detection unit) configured to conduct the classification processing to quantify the degrees (the intensities) of the barriers detected based on the image data.

The above-described wheelchair user support mapping system 1 can develop the recommended route (the movement plane) more precisely by quantifying the degrees (the intensities) of the barriers.

Moreover, in the above-described wheelchair user support mapping system 1, the data of the action history of the wheelchair user 2 (see FIG. 1) includes shot image data of a level difference as the barrier condition and acceleration data at the time of passage on the level difference with the wheelchair.

According to the above-described wheelchair user support mapping system 1, it is possible to accurately perceive the state of unevenness on the road surface and the degree of the level difference by using the actually measured acceleration data.

Moreover, in the above-described wheelchair user support mapping system 1, the first mobile terminal 3 includes the display unit 3a configured to display the recommended route R and the barrier image Ph.

According to the above-described wheelchair user support mapping system 1, the wheelchair user 2 can check the barrier image Ph together with the recommended route (the movement plan). Thus, the wheelchair user 2 can understand the locations and details of the barriers at a glance. In this way, the wheelchair user 2 can easily confirm adequacy of the recommended route (the movement plan).

Moreover, in the above-described wheelchair user support mapping system 1, any of the wheelchair user 2 and a person other than the wheelchair user 2 can confirm the barrier image Ph as well as the recommended route (the movement plan) in advance before the wheelchair user 2 starts a movement, by using the display unit 10a of the fixed terminal 10 provided independently of the display unit 3a of the first mobile terminal 3.

This makes it possible to confirm the adequacy of the recommended route (the movement plan) more sufficiently.

Although the embodiment of the present invention has been described above, it is to be understood that the present invention is not limited only to the above-described embodiment but can also be carried out in various manners.

The above-described embodiment is designed such that the multiple wheelchair users 4 other than the wheelchair user 2 are supposed to collect the barrier information. However, the present invention is not limited to this configuration. In this context, the barrier information may be collected by the wheelchair user 2, by using a vehicle-mounted camera mounted on an automobile or the like, by other pedestrians, and so forth.

Moreover, the barriers are not limited only to the thirteen attributes such as the road widths of the pathways as described in the embodiment. In this context, the barriers may also be classified into other attributes such as presence or absence of sidewalks, road constructions, temperature, humidity, and noise.

Moreover, the image data in the embodiment is assumed to be a video. However, the present invention is not limited to this configuration. In this context, the image data maybe any of a still image, a temperature map, a noise map, a humidity map, and the like.

Moreover, the individual barrier condition DB 8b (the action history storage unit) of the embodiment shown in FIG. 2 is configured to extract and store the barrier conditions, which constitute the criteria for passability and impassability, based on the action histories of the wheelchair user 2 (see FIG. 1).

However, the individual barrier condition DB 8b (the action history storage unit) constituting the present invention may also be configured to extract and store the barrier conditions based on action histories (not illustrated) of a wheelchair user other than the wheelchair user 2 (such as the wheelchair user 4 shown in FIG. 2).

The wheelchair user support mapping system 1 described above can output the recommended route (the movement plan) more adequately by supplementing the barrier conditions not experienced by the wheelchair user 2 (see FIG. 1) with the barrier conditions experienced by a different wheelchair user.

Moreover, the embodiment has described the configuration to output the recommended route (the movement plan) by causing any of the wheelchair user 2 (see FIG. 1) or the person other than the wheel chair user 2 (each of whom may be hereinafter simply referred to as a “user”) to input the point of departure D_{P} and the point of destination D_{S} to the first mobile terminal 3 or the fixed terminal 10. However, the present invention may also be configured to output the recommended route (the movement plan) by allowing the user to input a pass point on the map in addition to the point of departure D_{P} and the point of destination D_{S}.

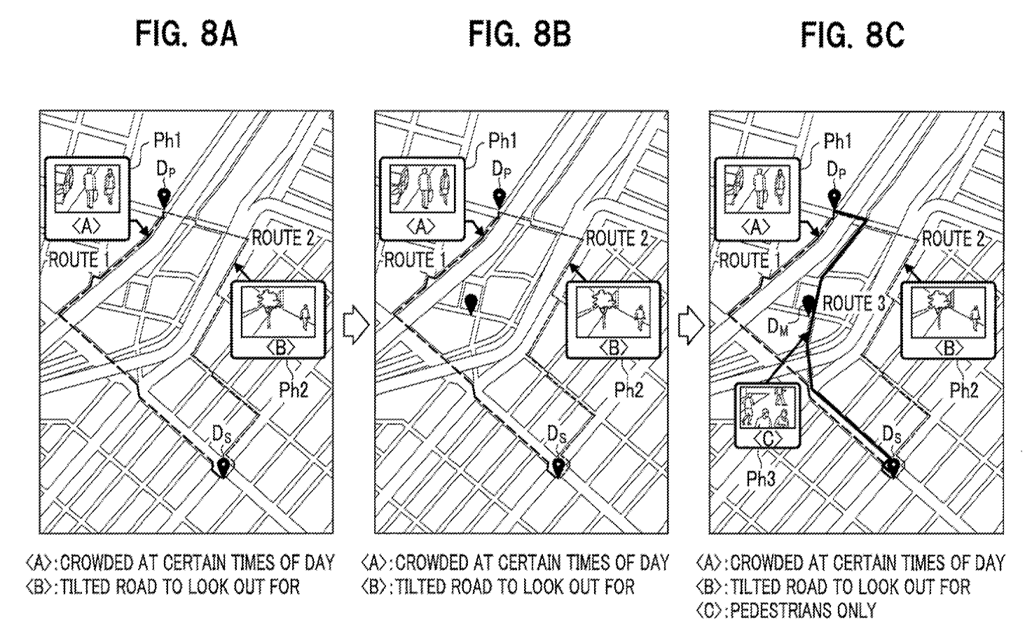

FIGS. 8A to 8C are diagrams of images on the display unit 3a or 10a (see FIG. 1) showing the progress from the input of the point of departure D_{P}, the point of destination D_{S}, and a pass point D_{M} to the output of the recommended route (the movement plan).

As shown in FIG. 8A, in the wheelchair user support mapping system 1 (see FIG. 1), a “route 1” and a “route 2” each connecting the point of departure D_{P} and the point of destination D_{S}, for example, are displayed on the display unit 3a or 10a (see FIG. 1) as the recommended routes (the movement plans) based on the point of departure D_{P} and the point of destination D_{S} input to the first mobile terminal 3 or the fixed terminal 10 (see FIG. 1) the by the user.

Moreover, barrier images Ph1 and Ph2 on the “route 1” and the “route 2” are displayed on the display unit 3a or 10a at the same time.

Moreover, the barrier images Ph1 and Ph2 may also include text messages such as “crowded at certain times of day” and “tilted road to look out for”.

When the user inputs the pass point D_{M} located between the point of departure D_{P} to the point of destination D_{S} to the first mobile terminal 3 or the fixed terminal 10 (see FIG. 1) as shown in FIG. 8B, a “route 3” that passes through the pass point D_{M} is displayed in addition to the “route 1” and the “route 2” on the display unit 3a or 10a as shown in FIG. 8C.

Moreover, the display unit 3a or 10a can additionally display a barrier image Ph3 or a text message concerning the “route 3”.

Here, the “route 3” that passes through the pass point D_{M} can be computed by use of an open API service adopting the above-described Web GIS, for example.

According to the above-described wheelchair user support mapping system 1 (see FIG. 1), it is possible to create the recommended route (the movement plan) while reflecting preferences of the user such as a place where the user wants to pass by and pathways that the user wants to use (such as pathways that allow the user to move as straight as possible).

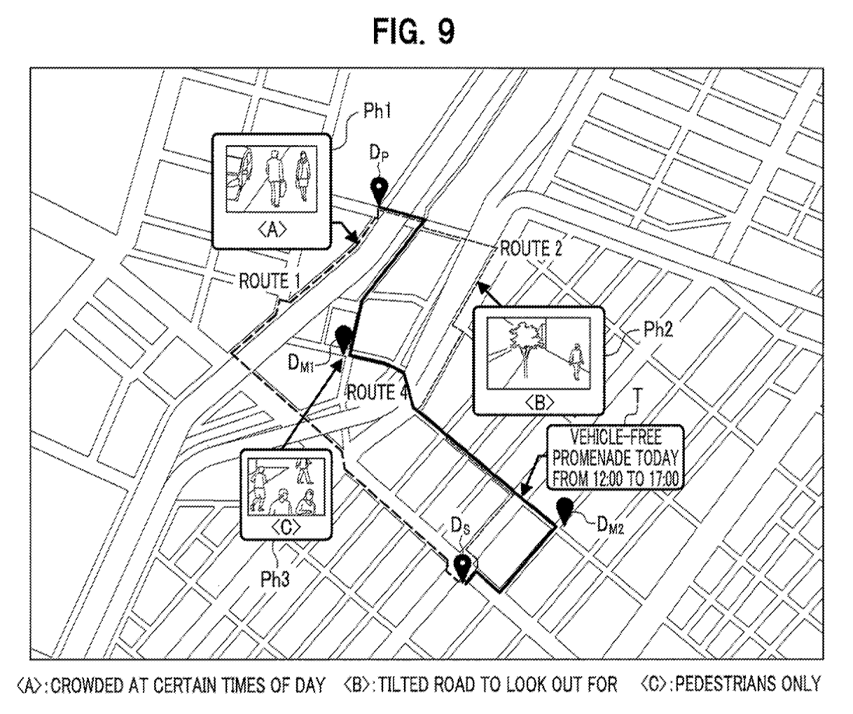

FIG. 9 is an image of the map showing an aspect in which the recommended route (the movement plan) is output after the input of the point of departure D_{P}, the point of destination D_{S}, a first pass point D_{M1}, and a second pass point D_{M2}.

As shown in FIG. 9, in this wheelchair user support mapping system 1 (see FIG. 1), the two pass points D_{M1} and D_{M2} are set on the way from the point of departure D_{P} to the point of destination D_{S}. Note that reference sign T in FIG. 9 denotes a text message.

According to the above-described wheelchair user support mapping system 1 (see FIG. 1), it is possible to create the recommended route (the movement plan) while reflecting the preferences of the user more in detail by setting the multiple pass points D_{M1} and D_{M2}.

Needless to say, it is possible to set three or more pass points.

Moreover, the above-described wheelchair user support mapping system 1 may also be configured to reflect a user evaluation, such as a feedback from the wheelchair user 2 (see FIG. 1) who has actually passed through the recommended route (see FIG. 2), in the next computation of the recommended route.

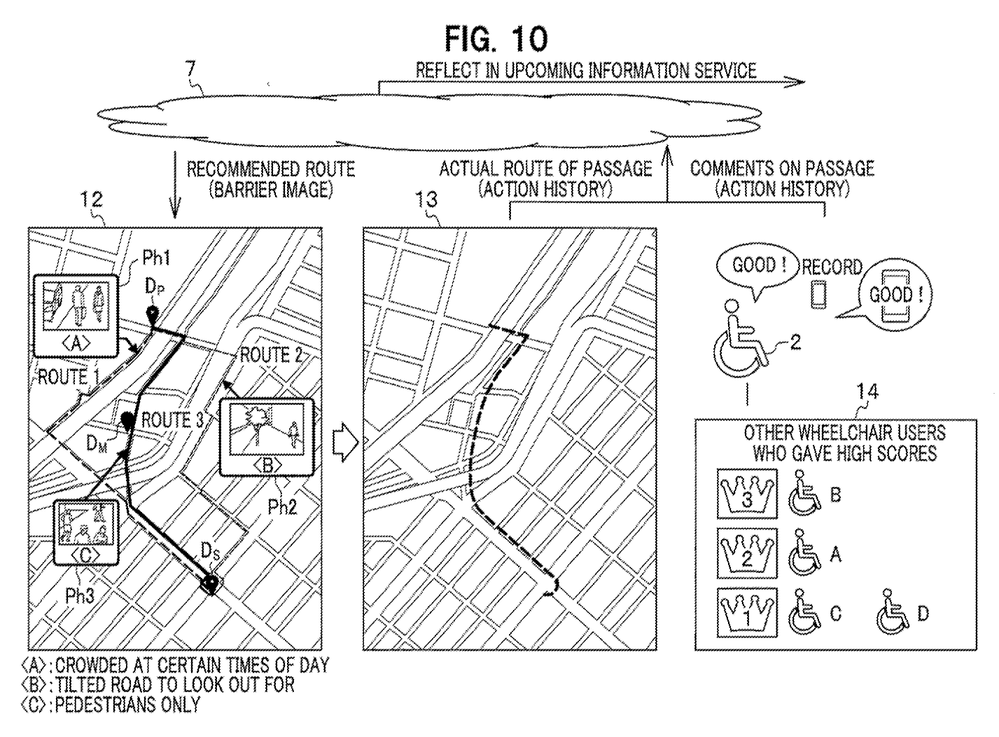

FIG. 10 is an explanatory configuration diagram of the wheelchair user support mapping system 1, which feeds back evaluations of the recommended route by users as the action histories of wheelchair user 2 (see FIG. 1).

As shown in FIG. 10, in this wheelchair user support mapping system 1, the cloud system 7 outputs the recommended route (the barrier images) in response to a request from the wheelchair user 2. The process to output the recommended route (the barrier images) is the same as the above-described process (see FIG. 2).

Then, a map image denoted by reference sign 12 in FIG. 10 is displayed on the display unit 3a (see FIG. 1) of the first mobile terminal 3 (see FIG. 1). The recommended routes including the “route 1”, the “route 2”, the “route 3”, and the like each connecting the point of departure D_{P} and the point of destination D_{S} that are input to the first mobile terminal 3 by the wheelchair user 2, the barrier images Ph1, Ph2, and Ph3, and the pass point D_{M} are displayed on this map image.

Next, when the wheelchair user 2 selects the “route 3” out of the three recommended routes and actually passes through the “route 3”, the trajectory of the “route 3” is displayed as an actual route of passage on the display unit 3a (see FIG. 1) of the first mobile terminal 3 (see FIG. 1) as illustrated in a map image denoted by reference sign 13 in FIG. 10. The “actual route of passage” is output to the cloud system 7 as an action history of the wheelchair user 2.

Moreover, the wheelchair user 2 inputs a feedback on passage of the recommended route, which the user has actually passed through, to the first mobile terminal 3 (see FIG. 1). The input of the feedback is assumed to be an input using a “like button” as found in an SNS (social networking service), an input according to a star rating (in a five-star scale), and the like. Here, the input of the feedback may also be carried out by a voice input to the smartphone by the wheelchair user 2, for example.

The above-described “feedback on passage” is also output to the cloud system 7 as an action history of the wheelchair user 2.

Then, the data of the “actual route of passage” and the “feedback on passage” are stored in the individual barrier condition DB 8b (see FIG. 2) and are reflected in the next computation of the recommended route.

Moreover, the feedback on the recommended route (the “feedback on passage”) by the wheelchair user 2 may also take the form of a rating by the wheelchair user 2 of feedbacks from wheelchair users other than the wheelchair user 2 who have passed through the recommended route. Specifically, assuming that there are four wheelchair users “A” to “D” other than the wheelchair user 2 as indicated in an image denoted by reference sign 14 in FIG. 10, for instance, an action history of “B” is weighted because “B” gave the highest score “3” in a range of score from 1 to 3.

Accordingly, the action history of “B” will be further reflected in the next computation of the recommended route.

Moreover, the embodiment has described the wheelchair user support mapping system 1 configured to output the route (the recommended route R) based on the barrier conditions applicable to the wheelchair user 2.

However, the present invention may also be configured to output the route while taking into account “favorite conditions” of the wheelchair user 2 in addition to the barrier conditions. Examples of the “favorite conditions” include surrounding scenery factors as typified by many plants, seaside roads, hillside roads, and the like. Nonetheless, the favorite conditions are not limited to the foregoing.

More specifically, let us assume a case where a route (1) which has less barriers but bad scenery and a route (2) which has good scenery but more barriers are selected in the state where there are several route candidates. In this case, the present invention may be configured to allow the wheelchair user 2 to select the route (2) in the first place and to move accordingly, and after the wheelchair user 2 is satisfied with the scenery, to change the route to the route (1) in midstream and to move accordingly. In other words, according to the present invention, it is possible to additionally input the pass point D_{M} so as to select the route (1) in midstream of the movement along the route (2) from the point of departure D_{P} to the point of destination D_{S}.

Moreover, the above-described embodiment assumes that the classification processing on the barriers is conducted by means of the image determination using the deep learning in the barrier quantification processing step S101 (see FIG. 3) executed by the barrier quantification processing unit 11 (the barrier detection unit).

Instead, the present invention may be configured to conduct the classification processing on the barriers by means of image determination using deep reinforcement learning which combines the deep learning and reinforcement leaning.

Here, the reinforcement learning has been known as a framework of learning control for learning a method of creating an operation signal to an environment such as a control target through a trial-and-error interaction with the environment so as to obtain a desirable measurement signal from the environment. In the reinforcement learning, the method of creasing the operation signal to the environment, with which an expected value of an evaluation value (a reward) to be obtained from a current state to the future is possibly maximized, is learned based on an evaluation value (a reward) of a scalar quantity to be calculated based on the measurement signal obtained from the environment.

As a consequence, according to the image determination using the above-described deep reinforcement learning, it is possible to achieve full automation control of the wheelchair user support mapping system 1 (see FIG. 1) by causing the wheelchair user support mapping system 1 to perform reward prediction as to which barrier should be presented in order to increase user satisfaction.

Although the embodiment of the present invention has been described and illustrated in detail, the disclosed embodiment is made for purposes of illustration and example only and not limitation. The scope of the present invention should be interpreted by terms of the appended claims.

### DESCRIPTION OF REFERENCE SIGNS

1: Wheelchair user support mapping system; 2: Wheelchair user; 3: First mobile terminal; 3a: Display unit; 4: Wheelchair user; 5: Second mobile terminal; 6: Third mobile terminal; 7: Cloud system; 8a: Barrier information DB (Association unit); 8b: Individual barrier condition DB (Action history storage unit); 9: Recommended route computation unit (Movement plan creation unit); 10: Fixed terminal; 10a: Display unit; 11: Barrier quantification processing unit (Barrier detection unit); D_{P}: Point of departure; D_{S}: Point of destination; Ph: Barrier image; R: Recommended route; S101: Barrier quantification processing step; S102: Barrier information accumulation step; S103: Individual barrier condition accumulation step; S104: Recommended route computation step

What is claimed is:

1. A wheelchair user support mapping system comprising:
an association unit configured to store actual image data of a location corresponding to a predetermined position on a map in such a way as to be capable of outputting the image data while associating the image data with the predetermined position on the map;
an action history storage unit configured to extract and store a barrier condition, which constitutes a criterion for passability and impassability, based on an action history of a wheelchair user; and
a movement plan creation unit configured to create a movement plan for the wheelchair user based on the barrier condition acquired with reference to the action history storage unit.

2. The wheelchair user support mapping system according to claim 1, further comprising
a barrier detection unit configured to quantify a degree of a barrier detected based on the image data.

3. The wheelchair user support mapping system according to claim 1, wherein
data of the action history include:
shot image data of a level difference as the barrier condition; and
acceleration data at the time of passage on the level difference with a wheelchair.

4. The wheelchair user support mapping system according to claim 1, wherein
the action history storage unit extracts and stores the barrier condition further based on an action history of a different wheelchair user from the wheelchair user, and
the movement plan creation unit creates the movement plan for the wheelchair user based on the barrier condition of the different wheelchair user from the wheelchair user acquired with reference to the action history storage unit.

5. The wheelchair user support mapping system according to claim 1, further comprising
a display unit configured to display the movement plan and the image data to the wheelchair user after the wheelchair user actually starts a movement based on the movement plan created by the movement plan creation unit.

6. The wheelchair user support mapping system according to claim 1, further comprising
a display unit configured to display the movement plan and the image data in advance before the wheelchair user starts a movement.

7. The wheelchair user support mapping system according to claim 2, further comprising
a display unit configured to display the movement plan and the image data to the wheelchair user after the wheelchair user actually starts a movement based on the movement plan created by the movement plan creation unit.

8. The wheelchair user support mapping system according to claim 2, further comprising
a display unit configured to display the movement plan and the image data in advance before the wheelchair user starts a movement.

9. The wheelchair user support mapping system according to claim 3, further comprising
a display unit configured to display the movement plan and the image data to the wheelchair user after the wheelchair user actually starts a movement based on the movement plan created by the movement plan creation unit.

10. The wheelchair user support mapping system according to claim 3, further comprising
a display unit configured to display the movement plan and the image data in advance before the wheelchair user starts a movement.

11. The wheelchair user support mapping system according to claim 4, further comprising
a display unit configured to display the movement plan and the image data to the wheelchair user after the wheelchair user actually starts a movement based on the movement plan created by the movement plan creation unit.

12. The wheelchair user support mapping system according to claim 4, further comprising
a display unit configured to display the movement plan and the image data in advance before the wheelchair user starts a movement.
